(19) United States
(12) Patent Application Publication
(10) Pub. No.: US 2020/0150007 A1
(43) Pub. Date: May 14, 2020

(54) SMEAR PLATE, SMEAR METHOD, AND SMEAR DEVICE

(71) Applicant: NOUL CO., LTD., Yongin-si, Gyeonggi-do (KR)

(72) Inventors: Dong Young LEE, Yongin-si, Gyeonggi-do (KR); Chan Yang LIM, Seongnam-si, Gyeonggi-do (KR); Kyung Hwan KIM, Yongin-si, Gyeonggi-do (KR)

(21) Appl. No.: 16/631,382

(22) PCT Filed: Jul. 17, 2017
(86) PCT No.: PCT/KR2017/007671

§ 371 Date: Jan. 15, 2020

(51) Int. Cl. G01N 1/28; G01N 33/487; C12M 1/12

(52) U.S. Cl. CPC G01N 1/2813; C12M 23/04; G01N 33/487

(57) ABSTRACT

The present disclosure provides a smear plate, a smear method, and a smear device. According to an embodiment of the present disclosure, the smear plate is for smearing a biological sample on an external substrate, the smear plate including: a body having a plate shape with a length, a width, and a pair of main surfaces, the body having a predetermined angle with respect to the external substrate; a support portion located on a side of the body and spaced apart from the external substrate by a predetermined distance; and a contact portion located on an opposite side of the body and including a pattern surface which is recessed inward of the body to induce fine particles included in the biological sample to move toward a midpoint in a width direction of the body for preventing the fine particles from being relatively densely distributed in outer regions in a width direction of the external substrate.

### TECHNICAL FIELD

The present disclosure relates to a smear method, a smear device, and a smear plate, and more particularly, to a smear plate for uniformly smearing a sample, and a method and device for smearing a sample using the smear plate.

### BACKGROUND ART

A blood smear test is a test method of smearing and staining blood and observing the morphology of blood cells using a microscope. The blood smear test is mainly used for diagnosing infections with parasitic diseases such as malaria, blood tumors including leukemia, and congenital blood cell abnormalities.

In particular, test methods for observing blood essentially require a process of smearing blood. However, since blood smearing is manually performed using a pair of slide glass plates in the related art, blood is unevenly smeared, and blood cells or blood is unevenly concentrated in outer areas.

Furthermore, in blood smear tests of the related art, the process of smearing, staining, and microscopic observation of blood is dependent on the inspectors manual work. Thus, when the inspector is not skilled, the smear of blood is uneven, and even when the inspector is skilled, the smeared state varies each time smearing is performed, thereby making it difficult to guarantee the reliability of smeared blood use for diagnosing diseases.

Therefore, it is required to develop a smear tool for uniformly smearing biological samples such as blood.

### DESCRIPTION OF EMBODIMENTS

### Technical Problem

An objective of the present disclosure is to provide a smear plate, a smear device, and a smear method.

Another objective of the present disclosure is to provide a smear plate, a smear device, and a smear method for smearing a sample on an external substrate.

Another objective of the present disclosure is to provide a smear plate, a smear device, and a smear method for smearing a sample while guaranteeing uniform distribution of particles included in the sample.

However, objectives of the present disclosure are not limited thereto, and additional objectives of the present disclosure will be set forth in part in the description and the accompanying drawings, and will be apparent from the description to those of ordinary skill in the related art.

### Solution to Problem

According to an embodiment of the present disclosure, there may be provided a smear plate for smearing a biological sample on an external substrate, the smear plate including: a body having a plate shape with a length, a width, and a pair of main surfaces, the body having a predetermined angle with respect to the external substrate; a support portion located on a side of the body and spaced apart from the external substrate by a predetermined distance; and a contact portion located on an opposite side of the body and including a pattern surface which is recessed inward of the body to induce fine particles included in the biological sample to move toward a midpoint in a width direction of the body for preventing the fine particles from being relatively densely distributed in outer regions in a width direction of the external substrate.

According to another embodiment of the present disclosure, a smear may include: an upper plate configured to support a smear plate at a predetermined downward inclination angle with respect to a first direction in which the biological sample is smeared, move in a second direction opposite the first direction to slide the smear plate for bringing the smear plate into contact with the biological sample, and move in the first direction to slide the smear plate such that the biological sample making contact with the smear plate is smeared in the first direction while following the smear plate; and a lower plate spaced downward from the upper plate by a predetermined distance and including a smear area in which the biological sample is placed and smeared.

According to another embodiment of the present disclosure, a smear method of smearing a biological sample in a smear area of an external substrate by using a smear plate, the method including: bring the smear plate into contact with the external substrate at a predetermined inclination angle with respect to the external substrate; sliding the smear plate on the external substrate in a first direction such that the smear plate is brought into contact with the biological sample placed at a predetermined position in the smear area and the biological sample is distributed along the smear plate; sliding the smear plate on the external substrate in a second direction opposite the first direction such that the biological sample is uniformly distributed in the second direction; and smearing the biological sample by using the smear plate while mitigating a phenomenon, in which fine particles included in the biological sample are relatively densely distributed in outer regions of the smear area in a width direction of the external substrate perpendicular to the second direction when the smear plate is slid in the second direction, by inducing the fine particles to move inward in the smear area along the contact area in the width direction of the external substrate, wherein an area of the smear plate making contact with the external substrate is concavely recessed inward of the smear plate.

However, aspects of the present disclosure are not limited thereto, and other aspects of the present disclosure will be apparent to those of ordinary skill in the art from the description and the accompanying drawings.

### Advantageous Effects of Disclosure

According to the present disclosure, samples may be uniformly smeared.

According to the present disclosure, the phenomenon in which particles included in samples are unevenly distributed in one direction may be mitigated.

According to the present disclosure, a sample may easily be smeared in one layer.

The effects of the present disclosure are not limited to the above-mentioned effects, and other effects not mentioned above may be clearly understood by those of ordinary skill in the art from the description and the accompanying drawings.

### BRIEF DESCRIPTION OF DRAWINGS

FIG. 1 is an example view illustrating a process of smearing a sample by a method of the related art.

FIG. 2 is an example view illustrating a process of smearing a sample by a method of the related art.

FIG. 3 is an example view illustrating a process of smearing a sample by a method of the related art.

FIG. 4 is an example view illustrating a uniformly smeared sample.

FIG. 5 is an example view illustrating an unevenly smeared sample.

FIG. 6 is an example view illustrating the case in which particles included in a sample are unevenly concentrated in outer areas.

FIG. 7 is a view illustrating the movement of particles included in a sample when the sample is smeared using a smear plate according to an embodiment of the present disclosure.

FIG. 8 is a view illustrating a smear plate according to an embodiment of the present disclosure.

FIG. 9 is a view illustrating a state in which a smear plate is deformed by contact with an external substrate according to an embodiment of the present disclosure.

FIG. 10 is a view illustrating a smear plate according to an embodiment of the present disclosure.

FIG. 11 is a view illustrating the inclination angle of the smear plate from an external substrate in the illustration of FIG. 10.

FIG. 12 is a view illustrating the inclination angle of a smear plate from an external substrate and the distribution of a sample between the smear plate and the external substrate according to an embodiment of the present disclosure.

FIG. 13 is a view illustrating an end surface of a smear plate according to an embodiment of the present disclosure.

FIG. 14 is a view illustrating a smear plate according to an embodiment of the present disclosure.

FIG. 15 is a view illustrating a smear plate according to an embodiment of the present disclosure.

FIG. 16 is a view illustrating a smear plate according to an embodiment of the present disclosure.

FIG. 17 is a view illustrating a fine pattern formed on an end surface of a smear plate according to an embodiment of the present disclosure.

FIG. 18 is a view illustrating a pattern of a plurality of recesses formed in an end surface of a smear plate according to an embodiment of the present disclosure.

FIG. 19 is a view illustrating a process of smearing a sample using a smear device according to an embodiment of the present disclosure.

FIG. 20 is a view illustrating a process of smearing a sample using a smear device according to an embodiment of the present disclosure.

FIG. 21 is a view illustrating a process of smearing a sample using a smear device according to an embodiment of the present disclosure.

FIG. 22 is a flowchart illustrating a smear method according to an embodiment of the present disclosure.

FIG. 23 is a schematic view illustrating a smear plate used for a smear experiment according to an embodiment of the present disclosure.

FIG. 24 is a view illustrating results of an experiment according to an embodiment of the present disclosure.

FIG. 25 is a view illustrating results of an experiment according to an embodiment of the present disclosure.

FIG. 26 is a view illustrating a sample smeared using a smear device and a smear plate according to an embodiment of the present disclosure.

FIG. 27 is a view illustrating a smear plate SP according to an embodiment of the present disclosure.

### BEST MODE

The embodiments described in the present disclosure are not intended to limit the scope of the present disclosure but are intended to clearly explain the idea of the present disclosure to those skilled in the art, and it should be construed that various modifications and variations could be made without departing from the scope of the present invention.

The terms used in the present disclosure are general terms currently widely used in the art in consideration of functions regarding the present disclosure, but the terms may vary according to the intention of those of ordinary skill in the art, precedents, or new technology in the art. Also, some terms may be arbitrarily defined, and in this case, the meaning of the terms will be described in the detailed description of the present disclosure. Thus, the terms used herein should not be construed based on only the names of the terms but should be construed based on the meaning of the terms together with the description throughout the present disclosure.

The drawings attached to the present specification are for easily explaining the present disclosure, and the shapes shown in the drawings may be exaggerated as necessary to help understanding of the present disclosure, and thus the present disclosure is not limited to the drawings.

Detailed descriptions related to well-known configurations or functions may be omitted in order not to unnecessarily obscure subject matters of the present disclosure.

According to an embodiment of the present disclosure, there may be provided a smear plate for smearing a biological sample on an external substrate, the smear plate including: a body having a plate shape with a length, a width, and a pair of main surfaces, the body having a predetermined angle with respect to the external substrate; a support portion located on a side of the body and spaced apart from the external substrate by a predetermined distance; and a contact portion located on an opposite side of the body and including a pattern surface which is recessed inward of the body to induce fine particles included in the biological sample to move toward a midpoint in a width direction of the body for preventing the fine particles from being relatively densely distributed in outer regions in a width direction of the external substrate.

An angle between a length direction of the body and a first tangent line, which is tangent to the pattern surface at a first point and is parallel to the main surfaces of the body, may be less than an angle between the length direction of the body and a second tangent line, which is tangent to the pattern surface at a second point and is parallel to the main surfaces of the body, and the second point may be closer to the midpoint in the width direction of the body than the first point is to the midpoint.

The pattern surface may include: a first surface located on a left side in the width direction of the body to guide the fine particles toward a right side in the width direction of the body; and a second surface located on the right side in the width direction of the body to guide the fine particles toward the left side in the width direction of the body.

The pattern surface may have a circular arc shape. The pattern surface may have a triangular shape with a vertex at the midpoint in the width direction of the body. The pattern surface may include a plurality of concave and convex portions.

When the smear plate is brought into contact with the external substrate and smears the biological sample, an inclination angle of at least a region of the contact portion from the external substrate may be different from an inclination angle of the support portion from the external substrate.

When the smear plate is brought into contact with the external substrate and smears the biological sample, an inclination angle of the contact portion from the external substrate may vary according to positions of the contact portion in the width direction of the body. An inclination angle of the contact portion from the external substrate may decrease in outward directions parallel to the width direction of the body.

The contact portion may be at least partially hydrophilic and may include a lower surface which is brought into contact with the biological sample. The body may have elasticity.

The biological sample may be blood, and the fine particles may be blood cells included in the blood.

According to another embodiment of the present disclosure, there may be provided a smear device for smearing a biological sample, the smear device including: an upper plate configured to support a smear plate at a predetermined downward inclination angle with respect to a first direction in which the biological sample is smeared, move in a second direction opposite the first direction to slide the smear plate for bringing the smear plate into contact with the biological sample, and move in the first direction to slide the smear plate such that the biological sample making contact with the smear plate is smeared in the first direction while following the smear plate; and a lower plate spaced downward from the upper plate by a predetermined distance and including a smear area in which the biological sample is placed and smeared. When the biological sample is smeared in the first direction, fine particles included in the biological sample may be separated from the smear plate in the second direction.

In this case, an end surface of the smear plate to be brought into contact with the smear area may be recessed in the first direction such that when the smear plate is brought into contact with the smear area and smears the biological sample in the first direction, outward movement of the fine particles included in the biological sample may be reduced.

An angle between a length direction of the body and a first tangent line, which is tangent at a first point to the end surface of the smear plate to be brought into contact with the smear area and is parallel to main surfaces of the body, may be less than an angle between the length direction of the body and a second tangent line, which is tangent at a second point to the end surface of the smear plate and is parallel to the main surfaces of the body, and the second point may be closer to a midpoint in a width direction of the body than the first point is to the midpoint.

Each of the smear plate and the smear area may have a length and a width, and the end surface of the smear plate to be brought into contact with the smear area may include: a first surface located on a left side in a width direction of the smear plate to guide the fine particles toward a right side in a width direction of the smear area; and a second surface located on the right side in the width direction of the smear plate to guide the fine particles toward the left side in the width direction of the smear area.

The end surface of the smear plate to be brought into contact with the smear area may include a plurality of concave and convex portions.

The smear plate may include a contact portion having an inclination angle varying with respect to the lower plate when being brought into contact with the lower plate, and when the smear plate is brought into contact with the smear area and smears the biological sample, the inclination angle of at least a region of the contact portion with respect to the external substrate may be different from an inclination angle of the support portion with respect to the external substrate.

When the smear plate is brought into contact with the smear area and smears the biological sample, an inclination angle of the contact portion from the external substrate may vary according to positions of the contact portion in a width direction of the body.

According to another embodiment of the present disclosure, there may be provided a smear method of smearing a biological sample in a smear area of an external substrate by using a smear plate, the method including: bring the smear plate into contact with the external substrate at a predetermined inclination angle with respect to the external substrate; sliding the smear plate on the external substrate in a first direction such that the smear plate may be brought into contact with the biological sample placed at a predetermined position in the smear area and the biological sample may be distributed along the smear plate; sliding the smear plate on the external substrate in a second direction opposite the first direction such that the biological sample may be uniformly distributed in the second direction; and smearing the biological sample by using the smear plate while mitigating a phenomenon, in which fine particles included in the biological sample are relatively densely distributed in outer regions of the smear area in a width direction of the external substrate perpendicular to the second direction when the smear plate is slid in the second direction, by inducing the fine particles to move inward in the smear area along the contact area in the width direction of the external substrate, wherein an area of the smear plate making contact with the external substrate may be concavely recessed inward of the smear plate.

The smear plate may have a length and a width. The smear plate may have a pair of main surfaces. In this case, the smear plate may include a pattern surface to control distribution of the fine particles on the external substrate, and the pattern surface may include a first point and a second point, wherein a first tangent line which is tangent to the pattern surface at the first point and is parallel to the main surfaces of the smear plate may have a smaller angle with respect to a length direction of the smear plate than a second tangent line which is tangent to the pattern surface at the second point and is parallel to the main surfaces of the smear plate, and the second point may be closer to a midpoint in a width direction of the body than the first point.

The pattern surface may include: a first surface located on a left side in a width direction of the smear plate to guide the fine particles toward a right side in the width direction of the smear plate; and a second surface located on the right side in the width direction of the smear plate to guide the fine particles toward the left side in the width direction of the smear plate. The pattern surface may include a plurality of concave and convex portions.

As an example of in vitro diagnosis, a medical sample may be collected and tested to diagnose various kinds of diseases or perform medical/non-medical practice such as checking the health status of the subject. In this case, the distribution or form of the components of the collected sample may be observed and tested according to an inspection method. For example, it may be necessary to observe an enlarged image of the components of a biological sample to inspect the morphology of blood components according to a hematological test method, or to check the presence or distribution of a specific protein (for example, an antigen or antibody) in the body fluid according to an immunological test method. To this end, in general, the sample is smeared prior to observation. Hereinafter, in the present specification, such a smear method and a smear device will be described.

### 1. SMEARING OF BIOLOGICAL SAMPLES

### 1.1 Significance

As described above, when it is intended to perform a test by observing a biological sample having liquid properties, a process of forming a thinly spread layer of the sample may be first performed for inspection of the sample using an image of the sample. This process of applying a sample to form a thin layer, preferably a single layer of the sample for ease of observation may be understood as smearing of a sample.

When smearing a sample, the uniformity of smearing and the degree of thinning of the smeared sample layer affect the ease of observation of the sample, the accuracy of sample-based diagnosis, or the like. Therefore, the smearing of a sample is required to be performed uniformly, thinly, and steadily.

### 1.2 Performing Smearing

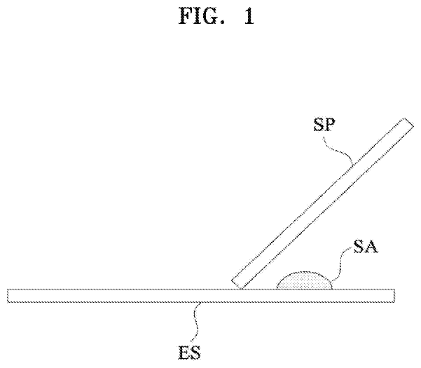

In general, the smearing of a sample may be performed using two plates. FIGS. 1 to 3 illustrate a process of smearing a sample SA using a first plate ES (that is, an external substrate ES) and a second plate SP (that is, a smear plate SP) in the related art. Hereinafter, smearing of the sample SA by a method of the related art will be described.

Referring to FIG. 1, in the method of the related art, smearing of the sample SA may include placing a small amount of the sample SA to be inspected on the first plate ES. Before the second plate SP is brought into contact with the sample SA, the sample SA may have a dome shape because of surface tension.

In addition, referring to FIG. 1, smearing of the sample SA according to the method of the related art may include bringing the second plate SP into contact with the first plate EP at a position slightly ahead of the sample SA placed on the first plate with a proper angle between the first plate SP and the second plate ES.

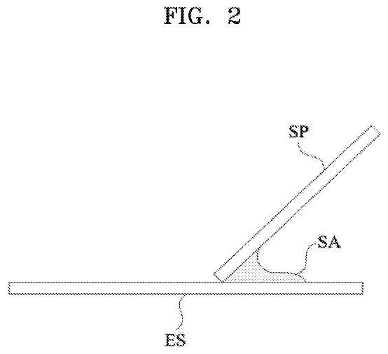

Referring to FIG. 2, the smearing of the sample SA according to the related-art method may include sliding the second plate SP backward on the first plate ES while maintaining the angle of the second plate SP to bring the second plate SP into contact with the sample SA. When the second plate SP is brought into contact with the sample SA, the sample SA may be dispersed to a contact area between the first plate ES and the second plate SP because of capillary force.

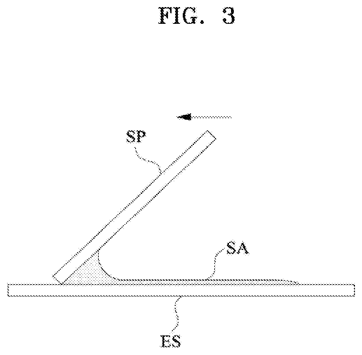

In addition, referring to FIG. 3, the smearing of the sample SA according to the related-art method may include smearing the sample SA on the first plate ES by pushing the second plate SP forward in contact with the first plate ES with an appropriate speed. When the second plate SP is slid forward in contact with the sample SA, the sample SA may be spread on the first plate ES because of attraction force between the second plate SP and the sample SA. As the sample SA is properly spread, a single layer of the sample SA may be formed.

1.3 Uniformity of Smeared Sample

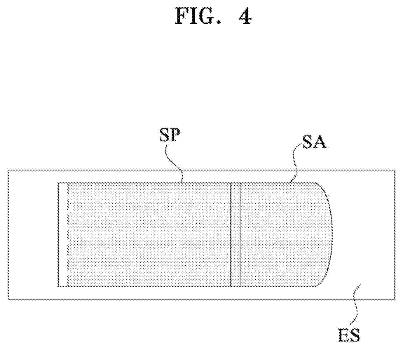

It is preferable that smearing the sample SA on the external substrate be performed uniformly not only in the smearing direction but also in a direction perpendicular to the smearing direction as shown in FIG. 4.

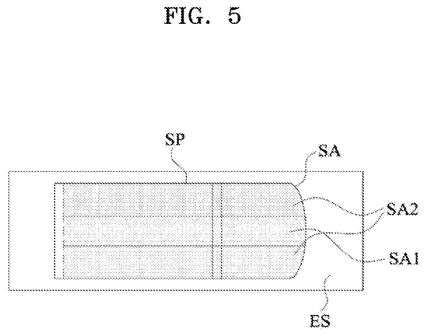

However, in the related-art method using a pair of plates as described above, when smearing a sample containing particles, the particles may be unevenly distributed in a direction perpendicular to the advancing direction (that is, smearing direction) of a plate SP used to apply the sample as shown in FIG. 5.

In other words, when the sample is applied using a pair of plates, the sample may be smeared on the first plate ES as shown in FIG. 5 in a state in which the density of the particles contained in the sample is greater at outer portions SA2 of the first plate ES in the width direction of the first plate ES than at a center portion SA1 of the first plate ES in the width direction of the first plate ES. As a specific example, in the case of smearing blood on a plate, relatively large white blood cells among the components of the blood may be arranged relatively densely at outer portions of the plate due to force pushing the white blood cells in directions perpendicular to the smearing direction.

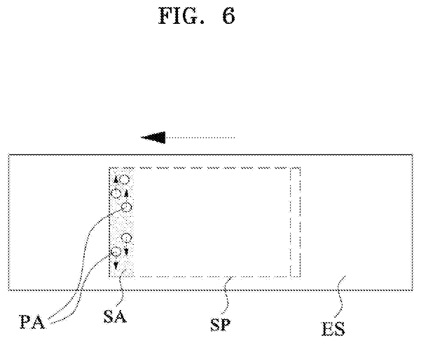

FIG. 6 is a view illustrating the case in which particles included in a sample are unevenly concentrated in outer areas. More specifically, FIG. 6 illustrates the movement of particles PA included in a sample SA when a smear plate SP is brought into contact with the sample SA placed on an external substrate ES and is then slid in the direction of the arrow. When the smear plate SP is brought into contact with the sample SA, the sample SA is distributed along an area in which the smear plate SP is in contact with the external substrate ES. Referring to FIG. 6, the particles PA included in the sample SA, which is distributed along a contact area with the external substrate ES, may be pushed by force acting in outward directions of the external substrate ES.

The force acting on the particles PA in the outward directions of the external substrate ES may be due to flows of a fluid generated as the smear plate SP is moved. More specifically, when the smear plate SP which is in contact with the sample is slid, fluid flows may occur in the outward directions of the external substrate ES in a rear area of the sample SA in the moving direction of the smear plate SP. The fluid flows may apply dragging force to the particles included in the sample SA such as white blood cells. Due to the fluid flows, the particles PA contained in the sample SA may be pushed in directions perpendicular to the sliding direction of the smear plate SP and may be relatively densely distributed in outer areas. The fluid flows may be understood as lateral flows.

The lower the density of particles, the more the fluid flows may have an influence on the particles. When the sample SA is blood, white blood cells having a lower density than red blood cells may be more significantly affected.

Uneven smearing described above does not serve the purpose of obtaining proper diagnostic results by uniformly applying the components of a sample to a plate, and thus a solution for this is required. In this regard, a smear plate, a smear method, a smear device, and the like for uniformly distributing particles contained in a sample will be described later.

### 1.4 Types of Biological Samples

Biological samples described in the present specification may be any biological samples which are in a suspension state and are used for diagnosing diseases. In particular, any biological sample in a suspension state may correspond to the biological sample of the present disclosure which is be applied to a substrate as an object to be observed, that is, which is an object to be smeared. Examples of the biological sample to be smeared may include blood, cell fluids, urine, and saliva. In addition, the biological sample may include fine particles. For example, white blood cells, red blood cells, or the like included in blood may correspond to the fine particles. The fine particles included in the biological sample may be microbeads or nanobeads. The fine particles may be protein particles. The fine particles may be biological particles such as antigens and antibodies.

Hereinafter, the present disclosure will be described based on a smear device, a smear method, and the like for smearing a biological sample on an external substrate. However, the smear device, the smear method, and the like of the present disclosure are not limited to being used for smearing a biological sample, but may be widely used for uniformly spreading a sample including small particles to uniformly distribute the particles.

### 2. SMEAR PLATE

### 2.1 Significance

Hereinafter, a smear plate used for uniformly smearing a biological sample will be described. The smear plate is a member which is brought into contact with a biological sample placed in an arbitrary smear area for smearing the biological sample in the smear area, and the smear plate sliding in the smear area may have a plate shape for uniformly spreading the biological sample.

### 2.2 Functions

According to an embodiment of the present disclosure, the smear plate may be manufactured in a special shape for uniformly smearing a biological sample on an external substrate. In other words, the smear plate may have a modified shape such that when the smear plate smears a biological sample on the external substrate, it may be possible to reduce the phenomenon in which fine particles included in the biological sample are relatively densely distributed in outer areas as the smear plate slides on the external substrate.

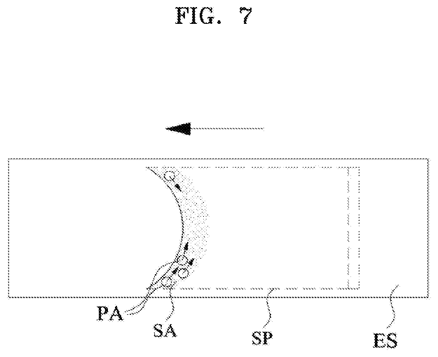

FIG. 7 is a view illustrating the movement of particles included in a sample when the sample is smeared using a smear plate according to an embodiment of the present disclosure. Like FIG. 6, FIG. 7 is a view illustrating the movement of particles PA included in a biological sample SA when a smear plate SP is brought into contact with the biological sample SA placed on an external substrate ES and slides in the arrow direction. Hereinafter, the function of the smear plate SP, which is designed to reduce the phenomenon in which the particles PA included in the sample are relatively densely distributed in outer areas, will be described with reference to FIG. 7 according to an embodiment of the present disclosure.

According to the embodiment of the present disclosure, an end surface of the smear plate SP provided on a side making contact with the external substrate ES may be inwardly recessed as shown in FIG. 7. In other words, the smear plate SP having a concave end surface may be provided such that the closer the contact point between the smear plate SP and the external substrate ES is to a midpoint of the external substrate SP, the more backward the contact point is in the smearing direction.

In this case, fluid flows may be generated such that the fine particles PA included in the biological sample SA, which is spread and smeared between the smear plate SP and the external substrate ES in a contact area in which the smear plate SP is in contact with the external substrate ES, may be directed toward the midpoint of the external substrate ES along the contact area between the smear plate SP and the external substrate ES. Therefore, force acting in outward directions on the particles PA may be canceled due to the sliding of the smear plate SP. That is, the phenomenon in which the fine particles PA are relatively densely distributed in outer areas of the external substrate ES by the sliding of the smear plate SP may be mitigated.

2.3 Form

As described above, a smeared sample in which particles are evenly distributed may be obtained by appropriately designing the end surface of the smear plate which makes contact with the external substrate. Here, the shape of the smear plate for performing uniform smearing will be described with reference to some embodiments.

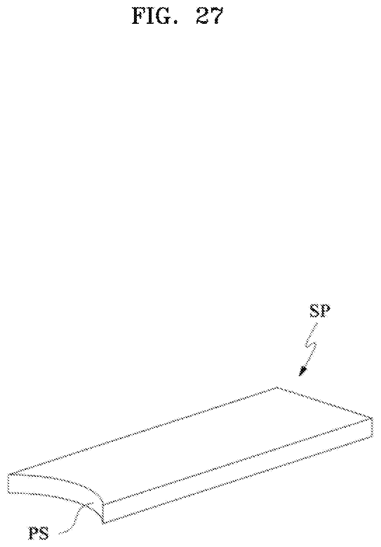

FIG. 27 is a view illustrating a smear plate SP according to an embodiment of the present disclosure. Referring to FIG. 27, the smear plate SP according to the embodiment of the present disclosure may have a pattern surface PS recessed inward of the smear plate SP. Referring to FIG. 27, the smear plate SP according to the embodiment of the present disclosure may have a structure in which a pair of main surfaces are distant by a predetermined distance. In FIG. 27, the pattern surface PS is inwardly recessed, but the pattern surface PS of the present disclosure is not limited thereto and may have various shapes as shown in the following embodiments and other various shapes inferable therefrom.

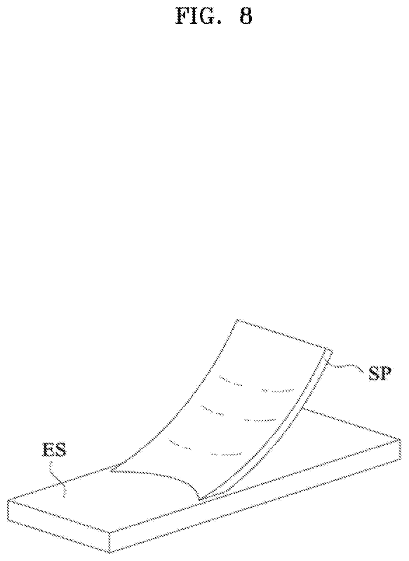

FIG. 8 is a view illustrating a smear plate SP according to an embodiment of the present disclosure. FIG. 8 is a schematic view illustrating an embodiment of the present disclosure in which when smearing is performed using the smear plate SP having an inwardly recessed end surface on a side making contact with an external substrate ES, the smear plate SP is deformed as a result of contact with the external substrate ES. As shown in FIG. 8, the smear plate SP may have a side which is fixed and spaced apart from the external substrate ES by a predetermined distance, and an opposite side which is brought into contact with the external substrate ES. In this case, the smear plate SP has to be brought into tight contact with the external substrate ES to apply capillary force to a sample in a contact area with the external substrate ES. When the distance between the side of the smear plate SP and the external substrate ES is properly determined, an edge at which the inwardly recessed end surface of the smear plate SP meets a lower surface of the smear plate SP may be brought into tight contact with the external substrate ES. Therefore, at least a portion of the smear plate SP may be deformed by vertical dragging force acting from the external substrate ES.

Hereinafter, an embodiment of a possible form of the smear plate SP and an embodiment of a deformed form of the smear plate as a result of contact with the external substrate ES will be described.

In addition, in the present specification, contact between objects, including contact between the smear plate SP and the external substrate ES, does not necessarily mean surface contact. In the present specification, contact between objects may mean that the objects are sufficiently adjacent to each other for performing a desired function.

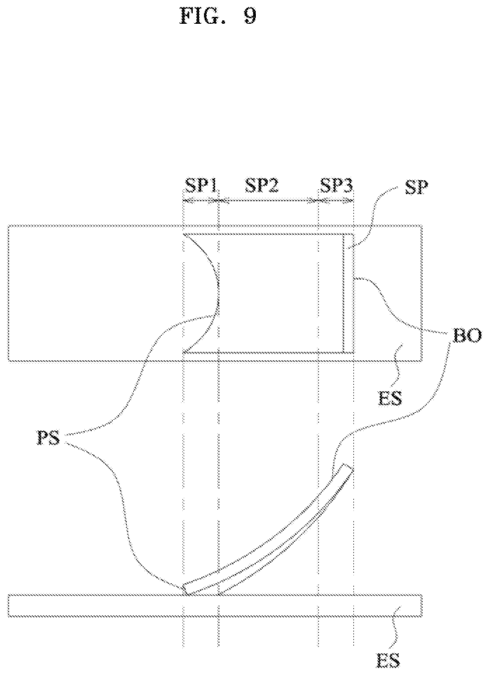

FIG. 9 is a view illustrating deformation of the smear plate SP caused by contact with the external substrate ES according to an embodiment of the present disclosure. FIG. 9 illustrates a plan view (upper region) and a side view (lower region) of the smear plate SP shown in FIG. 8.

Referring to FIG. 9, the smear plate SP may include a body BO, a contact portion SP1, a connection portion SP2, and a support portion SP3. The smear plate SP may be slid on the external substrate ES for smearing a biological sample on the external substrate ES. In this case, the biological sample may be previously dripped on the external substrate ES.

The body BO is provided in a plate shape having a length, a width, and a pair of main surfaces, and may be arranged at a predetermined inclination angle from the external substrate ES. For example, the body BO may have an inclination angle of 37° from the external substrate ES.

The support portion SP3 may be located at one side of the body BO and may be fixed and spaced apart from the external substrate ES by a predetermined distance. The support portion SP3 may be located at one side of the body BO that is far from the external substrate ES. The support portion SP3 may fix the body BO to a support unit which is spaced apart from the external substrate ES.

The contact portion SP1 may be located on the other side of the body BO, and may have a pattern surface which is inwardly recessed into the body BO such that fine particles included in the biological sample may be induced to move inward in the width direction of the body BO instead of being pushed outward in the width direction of the external substrate ES. The contact portion SP1 may have a hydrophilic lower surface making contact with the biological sample, that is, may have at least a portion of a surface making contact with the external substrate ES.

In the body BO, the connection portion SP2 may have a function of connecting the support portion SP3 and the contact portion SP1 to each other. In the body BO, the connection portion SP2 may be a portion which is not fixed and does not make contact with the external substrate ES. The connection portion SP2 may transfer power from the support portion SP3 to the contact portion SP1. For example, the smear plate SP may be slid on the external substrate ES as power transmitted to the support portion SP3 is transmitted to the contact portion SP1 through the connection portion SP2.

Although the contact portion SP1, the connection portion SP2, and the support portion SP3 are distinguished and individually described in the above embodiment, the contact portion SP1, the connection portion SP2, and the support portion SP3 of the body BO may be provided in one piece, and boundaries thereof may not be clear. In other words, although the body BO is divided into the contact portion SP1, the connection portion SP2, and the support portion SP3 in FIG. 9, the boundaries shown in FIG. 9 between the contact portion SP1, the connection portion SP2, and the support portion SP3 may not be always applied, and in some cases, the boundary between the contact portion SP1 and the connection portion SP2 of the body BO or the boundary between the connection portion SP2 and the support portion SP3 of the body BO may be ambiguous. Furthermore, in some cases, the connection portion SP2 may be included in the contact portion SP1 or the support portion SP3. Therefore, each portion should be determined according to the function thereof without regard to the boundaries shown in FIG. 9.

2.3.1 Contact Portion

According to an embodiment of the present disclosure, a smear plate may include a contact portion which has a pattern surface and is located on a side making contact with an external substrate. At least a portion of the contact portion may be deformed when being brought into contact with the external substrate. In this case, the degree of deformation of the shape of the contact portion may vary depending on the position in the width direction of the smear plate.

In other words, when the smear plate is brought into contact with the external substrate to smear a biological sample, the inclination angle of at least a portion of the contact portion from the external substrate may be different from the inclination angle of a support portion of the smear plate from the external substrate. In addition, when the smear plate is brought into contact with the external substrate to smear the biological sample, the inclination angle of the contact portion from the external substrate may vary depending on the position in a contact area in the width direction of a body of the smear plate. The inclination angle of the contact portion from the external substrate may decrease as it goes outward in the width direction of the body.

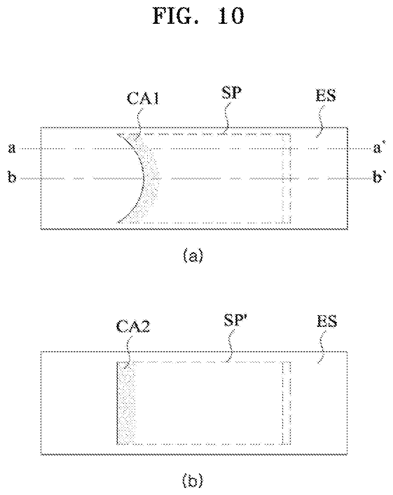

FIG. 10 is a view illustrating a smear plate SP according to an embodiment of the present disclosure. FIG. 10 is a view schematically illustrating the case (FIG. 10A) in which a sample is smeared using the smear plate SP according to an embodiment of the present disclosure as described above with reference to FIGS. 8 and 9, and the case (FIG. 10B) in which a sample is smeared using a smear plate SP′ manufactured by a method of the related art.

When smearing is performed using the smear plate SP having a concave end surface on a side making contact with an external substrate ES (FIG. 10A), the smear plate SP may be brought into tight contact with the external substrate ES and may thus form a contact area CA1. In this case, the sample to be smeared may be distributed along the contact area CA1. In other words, as shown in FIG. 10, the contact area CA1 may be biased in the smearing direction of the sample at outer sides of the external substrate ES, rather than at a center portion of the external substrate ES. In other words, the contact area CA1 may be thicker at the outer sides of the external substrate ES than at the center portion of the external substrate ES. Therefore, particles included in the sample may be pushed along the contact area toward the center portion of the external substrate ES. That is, as described with reference to FIGS. 6 and 7, an effect of mitigating non-uniform distribution of fine particles included in the sample may be obtained.

In addition, even when smearing is performed using the smear plate SP′ which is manufactured by a method of the related art and has a flat end surface (FIG. 10B), the smear plate SP′ may be brought into tight contact with the external substrate ES and may form a contact area CA2. In this case, the sample to be smeared may be distributed along the contact area CA2. The contact area CA2 may be uniform in the width direction of the external substrate ES, unlike the contact area CA1 formed in the above embodiment (FIG. 10A). In other words, the contact area CA2 may be uniform with a constant thickness at the center portion of the external substrate ES and at the outer sides of the external substrate ES. Therefore, as described with reference to FIGS. 6 and 7, when the smear plate SP′ is slid, fine particles included in the sample may be unevenly distributed.

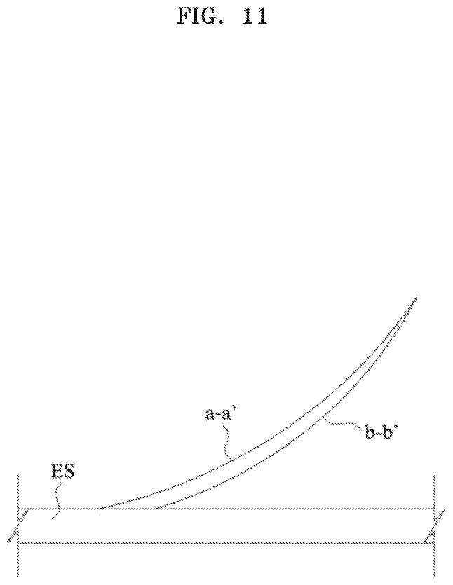

FIG. 11 is a view illustrating the inclination angle of the smear plate SP from the external substrate ES in the illustration of FIG. 10. FIG. 11 is a schematic cross-sectional view illustrating the smear plate SP with respect to two different positions of the external substrate ES in the width direction of the external substate in the illustration in FIG. 10. More specifically, lines connecting midpoints in the height direction of the smear plate SP with respect to two different positions in the width direction of the external substrate ES are shown to observe the inclination angle of the smear plate SP.

Referring to FIG. 11, the smear plate SP may have a cross section bent according to the external substrate ES in a plane (hereinafter, referred to as an a-a′ plane) passing through a line a-a′ shown in FIG. 10A and perpendicular to the external substrate ES. In addition, the smear plate SP may have a cross section bent according to the external substrate ES in a plane (hereinafter, referred to as a b-b′ plane) passing through a line b-b′ shown in FIG. 10A and perpendicular to the external substrate ES. In this case, the degree of bending of the smear plate SP in the a-a′ plane may be greater than the degree of bending of the smear plate SP in the b-b′ plane. In other words, since the smear plate SP has an inwardly recessed contact end, the length of the smear plate SP may be greater in the a-a′ plane than in the b-b′ plane. Since the other end of the smear plate SP is spaced apart from the external substrate ES by a predetermined distance and is fixed in parallel with the external substrate ES without making contact with the external substrate ES, force applied to the smear plate SP from the external substrate ES may be greater in the a-a′ plane than in the b-b′ plane.

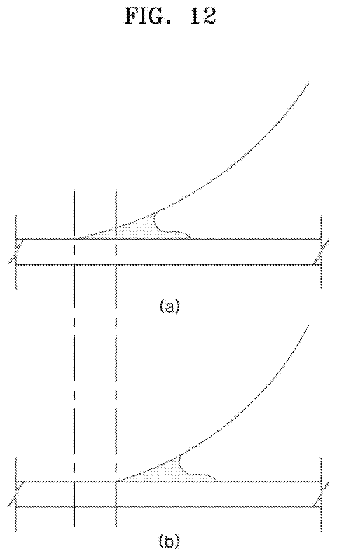

FIG. 12 is a view illustrating the inclination angle of the smear plate SP from the external substrate ES which is described with reference to FIG. 10 and the distribution of a sample between the smear plate SP and the external substrate ES. FIG. 12A schematically shows the cross-section of the smear plate SP in the a-a′ plane shown in FIG. 10 and the distribution of the sample in the a-a′ plane. FIG. 12B schematically shows the cross-section of the smear plate in the b-b′ plane shown in FIG. 10 and the distribution of the sample in the b-b′ plane.

Referring to FIG. 12, the smear plate SP and the external substrate ES may be obliquely in contact with each other. In this case, due to the shape of the smear plate SP, the point, at which the smear plate SP is in contact with the external substrate ES in the a-a′ plane which is a relatively outer plane, may be ahead of the point, at which the smear plate SP is in contact with the external substrate ES in the b-b′ plane. Therefore, the distribution of the sample between the external substrate ES and the smear plate SP may be different in the a-a′ plane and the b-b′ plane. For example, an area in which the sample is distributed in the a-a′ plane may be more biased in the smearing direction than an area in which the sample is distributed in the b-b′ plane. In addition, the area in which the sample is distributed in the a-a′ plane may have a greater length in the smearing direction than the area in which the sample is distributed in the b-b′ plane.

The sample may be distributed between the external substrate ES and the smear plate SP because both the external substrate ES and the smear plate SP attract the sample and capillary force is applied to the sample by a narrow gap between the external substrate ES and the smear plate SP. In this case, the external substrate ES and the smear plate SP may be in contact with each other at an oblique angle, and the sample may have an open surface on the opposite side in the smearing direction. The open surface may be a concave surface (meniscus) due to surface tension. The sample may flow in a space surrounded by the external substrate ES, the smear plate SP, and the open surface.

2.3.2 Pattern Surface

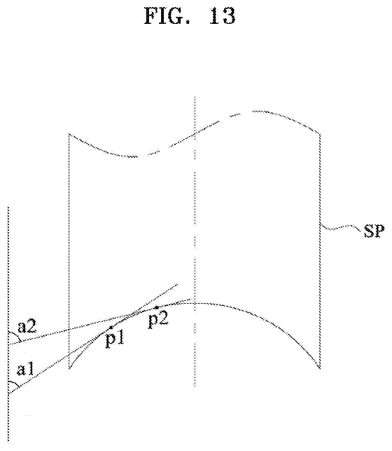

FIG. 13 is a cross-sectional view illustrating a smear plate SP according to an embodiment of the present disclosure. The smear plate SP may include a body and a contact portion formed on one side of the body as described above. The contact portion may have a pattern surface recessed inward of the body. FIG. 13 illustrates the pattern surface according to an embodiment of the present disclosure.

Referring to FIG. 13, the pattern surface has a first point p1 and a second point p2 selected at different positions on an end surface of the smear plate SP in the width direction of the smear plate SP (that is, an end surface parallel to main surfaces of a body of the smear plate SP). The second point p2 may be closer to a midpoint of the body in the width direction of the body than the first point p1. In this case, a first tangent line which is tangent to the pattern surface at the first point p1 and is parallel to the main surfaces of the body may have a smaller angle from the length direction of the body than a second tangent line which is tangent to the pattern surface at the second point p2 and is parallel to the main surfaces of the body.

In other words, the first point p1 located on the pattern surface may be more distant from a centerline of the body in the width direction of the body than the second point p2 located on the pattern surface.

The angle from the length direction of the body to a tangent line which is tangent to the end surface at a given point and is parallel to the main surfaces of the body may continuously vary as the given point approaches the midpoint of the body as shown in FIG. 13. Alternatively, the angle from the length direction of the body to the tangent line which is tangent to the end surface at the given point and is parallel to the main surfaces of the body may vary continuously in a certain section but may not vary in another section.

The pattern surface may include: a first surface located on the left side in the width direction of the body to guide fine particles toward the right side in the width direction of the body; and a second surface located on the right side in the width direction of the body to guide fine particles toward the left side in the width direction of the body.

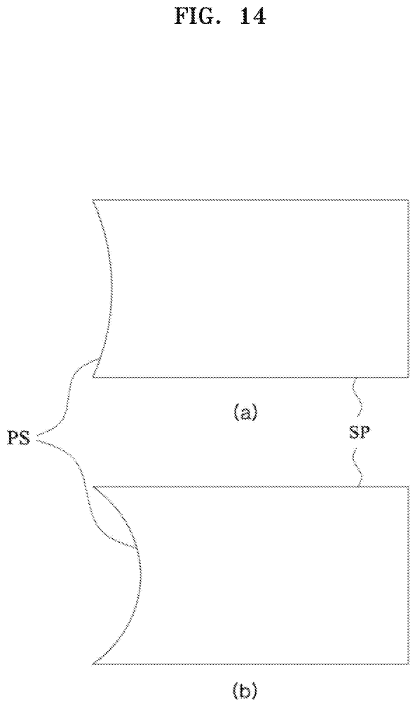

FIGS. 14 to 16 illustrate smear plates SP according to some embodiments of the present disclosure. The smear plates of the embodiments of the present disclosure may each include a contact portion formed at one side. Hereinafter, pattern surfaces PS formed on the contact portions at sides of the smear plates SP for uniformly smearing samples will be described according to embodiments. In the following description, a pattern surface PS having a predetermined shape may be understood that the pattern surface PS has an end surface of the shape. In addition, although the depth of the pattern surface PS recessed inward of the smear plate SP is exaggerated in each of FIGS. 14A to 16B to clearly illustrate the shape of the smear plate SP, it is preferable that the depth of the pattern surface PS recessed inward of the smear plate SP be set to be a reference depth or less for properly bringing the smear plate SP into tight contact with an external substrate.

Referring to FIG. 14, the pattern surface PS may be formed as a curved surface. The pattern surface PS may have a circular arc shape. The pattern surface PS may have variously circular arc shapes having different curvatures as shown in FIGS. 14A and 14B. In this case, the curvature of the pattern surface PS may be determined in consideration of the type and viscosity of a sample to be smeared, the purpose of smearing, and the like.

The pattern surface PS may be formed in a circular arc shape having one center point, or may be formed in a combination of circular arc shapes having a plurality of center points. In addition, when the pattern surface PS has a circular arc shape, the center point of the curved surface may be located on the same plane as the smear plate SP, or the center point of the curved surface may be located on a plane spaced a predetermined distance from the smear plate SP. In other words, the pattern surface PS having a circular arc shape may be perpendicular or oblique to the smear plate SP. In addition, the pattern surface PS may have an end surface with a cycloid curve shape.

In FIG. 14, only the case in which the smear plate SP has an circular arc-shaped pattern surface PS of which the center is located outside the smear plate SP is illustrated. However, if necessary, the smear plate SP may be formed to have a circular arc-shaped pattern surface PS of which the center is located inside the smear plate SP.

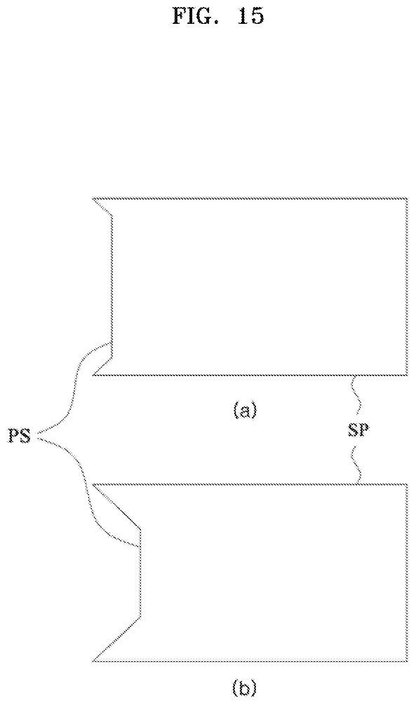

Referring to FIG. 15, the pattern surface PS may have a blunt triangular shape. The pattern surface PS may have a trapezoidal shape. As shown in FIGS. 15A and 15B, the depth of the pattern surface PS recessed inward of the smear plate SP may be differently determined in consideration of the type of a sample to be smeared. Furthermore, although the trapezoidal pattern surface PS is illustrated as having sharply bent corners in FIG. 15, the corners of the pattern surface PS may be smooth round corners for stable smearing of a sample.

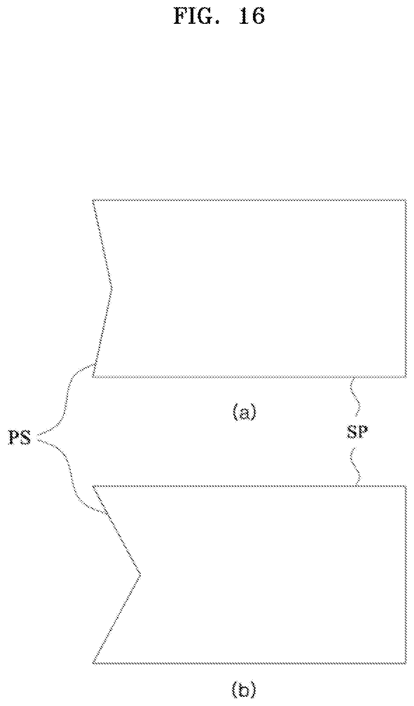

Referring to FIG. 16, the pattern surface PS may have a triangular shape having a vertex at a center point of the body in the width direction. As shown in FIGS. 16A and 16B, the height of the triangular shape of the pattern surface PS of the smear plate SP may be determined differently in consideration of the type of a sample to be smeared. Although the triangular pattern surface PS is illustrated in FIG. 16 as being sharply bent and thus having a vertex, the vicinity of the vertex of the pattern surface PS may be formed as a smooth round corner for stably smearing of a sample.

However, the pattern surfaces of the smear plates of the present disclosure are not limited to the embodiments shown in FIGS. 14 to 16, and it should be construed that the scope of the present disclosure includes combinations of the above-described embodiments and various other embodiments of smear plates having concave ends which are designed to prevent particles included in a sample from being pushed outward during smearing.

2.3.3 Surface

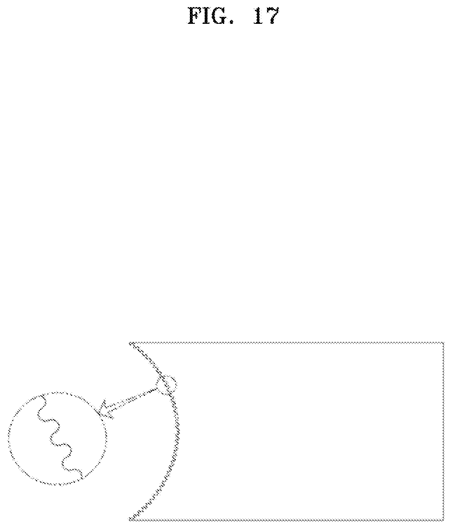

FIG. 17 is a view illustrating a fine pattern formed on an end surface of a smear plate according to an embodiment of the present disclosure. Referring to FIG. 17, the fine pattern including a plurality of concave and convex portions may be formed on the pattern surface according to the embodiment of the present disclosure.

The fine pattern may be formed to offset the flow of a fluid which is generated behind the smear plate in the moving direction of the smear plate when the smear plate is slid in one direction. The fine pattern may be formed by a plurality of grooves. The fine pattern may be formed as a dot pattern including a plurality of dots formed in the smear plate. The fine pattern may be formed as a wave pattern. The fine pattern may be formed in the form of a repeating cycloid curve. The fine pattern may be formed as a pattern including a plurality of repeating arcs.

In addition, although the smear plate illustrated as an example in FIG. 17 has a fine pattern surface which is inwardly recessed in a curved shape, the fine pattern may be applied to pattern surfaces of various forms.

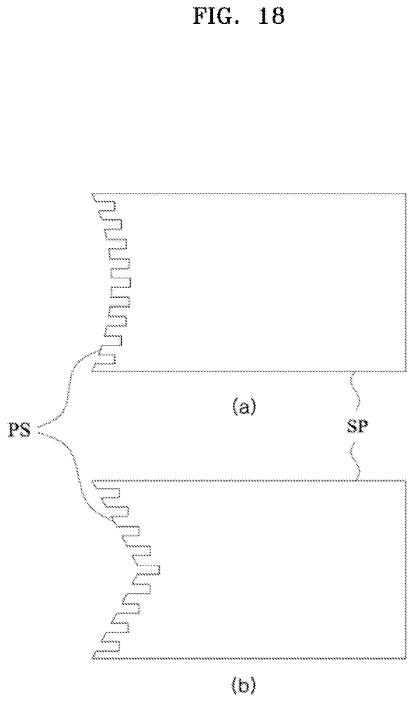

FIG. 18 is a view illustrating a pattern of a plurality of recesses formed in an end surface of a smear plate according to an embodiment of the present disclosure. Referring to FIG. 18, a pattern surface according to an embodiment of the present disclosure may have a recess pattern formed by a plurality of grooves formed in the smear plate.

As shown in FIG. 18, the recess pattern may include a plurality of rectangular grooves. However, the present disclosure is not limited thereto, and the recess pattern may include a plurality of triangular grooves or curved grooves. In addition, although FIG. 18 illustrate a smear plate having an inwardly recessed curved pattern surface or an inwardly recessed triangular pattern surface as an example, the recess pattern may be applied to pattern surfaces of various forms.

2.4 Material

According to an embodiment of the disclosure, a smear plate may be formed of a material having elasticity. When the smear plate is slid on the external substrate to smear a sample on an external substrate, the smear plate and the external substrate are required to apply appropriate pressure to each other in a direction perpendicular to the external substrate. In addition, a region in which the smear plate is brought into contact with the external substrate may be partially deformed to form a contact area in which the sample is distributed, and to this end, the smear plate may be formed of an elastic material.

According to an embodiment of the disclosure, the smear plate may be formed of a hydrophilic material. Alternatively, the smear plate may be coated with a hydrophilic material. Since most biological samples which are smeared according to the present disclosure are hydrophilic liquid substances or hydrophilic suspensions, the smear plate may be formed of a hydrophilic material for making contact with such a biological sample and smear the biological sample while sliding on the biological sample. The smear plate may be partially coated with a hydrophilic material. More specifically, a hydrophilic coating may be formed on a center portion of the smear plate in the width direction of the smear plate for uniform spreading of a biological sample.

Furthermore, in the present specification, the case in which a plate member having a length and a width is provided as a smear plate is described as a main example, but the present disclosure is not limited thereto. In some cases, a smear film having a thin thickness or a smear block having a predetermined thickness may be used as a smear plate. Hereinafter, a smear device which perform smearing using a smear plate will be described.

### 3. SMEAR DEVICE

### 3.1 Significance

In laboratories and places where diagnosis is performed, the above-described smearing of a sample is generally carried out by a skilled expert such as a researcher, a doctor, or the like. When smearing is performed by a person as described above, the spreading rate of samples may not be constant every time the smearing is performed, and the smearing may be unevenly performed because force is not uniformly applied to a smear plate. Thus, mechanization and automation of smear systems are required to obtain more uniformly and consistently smeared samples.

Hereinafter, a smear device for uniformly smearing a sample using a smear plate will be described. The smear device described in the present disclosure may be manufactured in the form of a disposable kit which is usable just once, or in the form of a reusable device of which some components are replaceable.

### 3.2 Configuration of Device

According to an embodiment of the present disclosure, a smear device may include an upper plate and a lower plate. According to an embodiment of the disclosure, the upper plate and the lower plate may have rectangular plate shapes corresponding to each other. According to another embodiment of the present disclosure, the upper plate and the lower plate may be fabricated to correspond to each other and may have disk shapes having a common center axis.

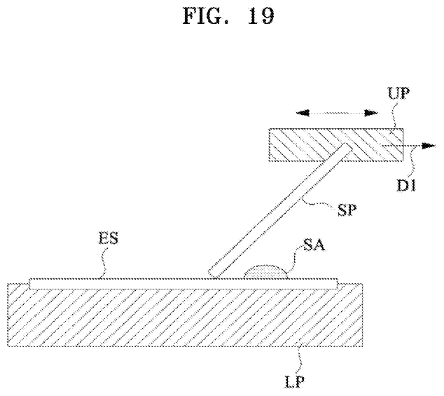

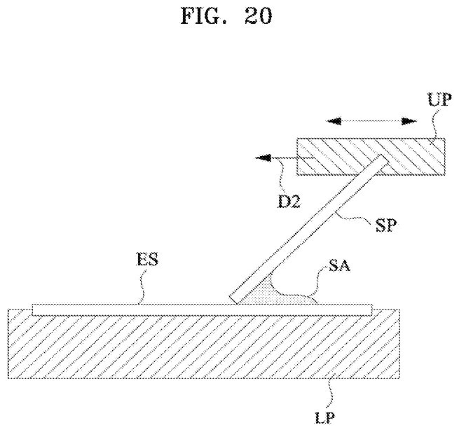

FIGS. 19 to 21 are views illustrating a smear device according to an embodiment of the present disclosure. Referring to FIGS. 19 to 21, the smear device of the embodiment of the present disclosure may include an upper plate UP and a lower plate LP. The upper plate UP may include a smear plate SP that is supported by the upper plate UP and is brought into contact with the lower plate LP to smear a sample SA. The upper plate UP may be provided to be movable in a length direction of the upper plate UP. The lower plate LP may include a smear area ES (external substrate ES) which makes contact with the smear plate SP. Hereinafter, each element of the smear device will be described with reference to FIGS. 19 to 20.

### 3.2.1 Upper Plate UP

The upper plate UP may support the smear plate SP in a state in which the smear plate SP is inclined downward by a predetermined angle with respect to a first direction in which a biological sample SA will be smeared, move in a second direction opposite to the first direction to slide the smear plate SP and bring the smear plate SP into contact with the biological sample SA, and move in the first direction such that the biological sample SA which is in contact with the smear plate SP is smeared in the second direction while following the smear plate SP.

The upper plate UP may be arranged in parallel to the lower plate LP (described later) and may be fixed. The upper plate UP may be moved in a direction parallel to the lower plate LP. The upper plate UP may be moved in a direction parallel to the lower plate LP such that the smear plate SP supported by the upper plate UP may be slid on the lower plate LP.

The first direction and the second direction in which the upper plate UP is moved may be linear directions or rotation directions. In other words, the expression “the upper plate UP is moved in the first direction” may mean that the upper plate UP is moved from the left side to the right side, and the expression “the upper plate UP is moved in the second direction” may mean that the upper plate UP is moved from the right side to the left side. In addition, the expression “the upper plate UP is moved in the first direction” may mean that the upper plate UP is rotated clockwise about a certain rotation axis, and the expression “the upper plate UP is moved in the second direction may mean that the upper plate UP is rotated counterclockwise about a certain rotation axis.

The movement of the upper plate UP may be a movement relative to the lower plate LP (described later). In other words, the upper plate UP may be moved in the first or second direction relative to the lower plate LP. However, the present disclosure is not necessarily limited thereto, and the moving direction of the upper plate UP may refer to a direction suitable for sliding the smear plate SP in a smear area on the lower plate LP.

The upper plate UP may further include a vertically movable member which is vertically movable to place a biological sample SA close to the smear area. The upper plate UP may be moved such that the biological sample SA may be brought into contact with the smear area. For example, when a smearing process using the smear plate SP is completed, the upper plate UP may be moved relative to the smear area of the biological sample SA and may perform a contact operation for a post treatment in the smear area. For example, the upper plate UP may be vertically moved and brought into contact with the smear area of the biological sample SA to supply a fixative for fixing the biological sample SA to the smear area.

The upper plate UP may be moved at a predetermined speed. The predetermined speed may be determined based on the viscosity of the biological sample SA, the type of the biological sample SA, the temperature of the biological sample SA, the type of biological particles contained in the biological sample SA, the downward inclination angle of the smear plate SP from the upper plate UP, the depth of a pattern surface of the smear plate SP, or the like. For example, the moving speed of the upper plate UP may be decreased as the viscosity of the biological sample SA increases. In addition, the moving speed of the upper plate UP may be increased as the degree of concavity of the pattern surface of the smear plate SP increases. However, the present disclosure is not limited thereto, and the moving speed of the upper plate UP may be increased or decreased according to the purpose of smearing of the biological sample SA.

The upper plate UP may be provided in a state in which the upper plate UP is fixed at a position spaced upward from the smear area of the biological sample SA by a predetermined distance. The predetermined distance between the upper plate UP and the smear area may be determined by considering the downward inclination angle of the smear plate SP fixed to the upper plate UP and the protruding length of the smear plate SP from the upper plate UP. In other words, the predetermined distance between the upper plate UP and the smear area of the biological sample SA may be determined such that an end of the smear plate SP may be brought into tight contact with the lower plate LP with a proper contact force. If the distance between the upper plate UP and the lower plate LP is too small, the friction between the smear plate SP and the lower plate LP may be too strong to properly perform smearing, and if the distance is too large, a single layer of the biological sample SA may not be formed. Therefore, the upper plate UP and the lower plate LP may be required to be spaced apart from each other by a proper distance.

The upper plate UP may further include a dripping unit for dripping a biological sample SA on the smear area of the biological sample SA. The upper plate UP may drip the biological sample SA on the smear area of the biological sample SA at a position which is spaced a predetermined distance in the first direction from a position at which the smear plate SP is in contact with the smear area.

The upper plate UP may be manufactured such that the smear plate SP supported by the upper plate UP is detachably attached to the upper plate UP. For example, even when the smear device is manufactured to be used multiple times, the smear plate SP supported by the upper plate UP may be replaced with a new smear plate SP each time the biological sample SA is replaced with a new biological sample SA.

In addition, an end surface of the smear plate SP may have an inwardly recessed shape such that when the smear plate SP supported by the upper plate UP is brought into contact with the smear area and the smear plate SP smears the biological sample SA in the first direction, outward movements of fine particles of the biological sample SA caused by the smearing of the sample SA may be reduced. The smear plate SP may have a variety of shapes and may be brought into contact with the lower plate LP to smear the biological sample SA, and the above-described details on the smear plate SP may be applied throughout the present specification.

For example, the smear plate SP supported by the upper plate UP may include a contact end surface for contact with the smear area located at a lower side. In this case, a first tangent line, which is tangent at a first point to the contact end surface of the smear plate SP for contact with the smear area and is parallel to main surfaces of the smear plate SP, may have a smaller angle from the length direction of a body of the smear plate SP than a second tangent line, which is tangent at a second point to the contact end surface of the smear plate SP and is parallel to the main surfaces of the smear plate SP (the second point is closer to a midpoint of the body in the width direction of the body than the first point is to the midpoint).

In addition, the smear plate SP and the smear area may each have a length and a width, and the end surface of the smear plate SP for contact with the smear area may include: a first surface located on the left side in the width direction to guide fine particles toward the right side in the width direction; and a second surface located on the right side in the width direction to guide fine particles toward the left in the width direction.

In addition, the smear plate SP may include a contact portion which makes contact with the lower plate LP and has a variable inclination angle with respect to the lower plate LP. When the smear plate SP is brought into contact with the smear area and smears the biological sample SA, the inclination angle of at least a portion of the contact portion from an external substrate may be different from the inclination angle of a support portion of the smear plate SP from the external substrate. In addition, when the smear plate SP is brought into contact with the smear area and smears the biological sample SA, the inclination angle of the contact portion with respect to the external substrate may vary according to the position of the contact portion in the width direction of the body of the smear plate SP.

### 3.2.2 Lower Plate LP

The lower plate LP may be spaced apart from the upper plate UP in a downward direction by a predetermined distance and may include the smear area in which the biological sample SA is placed and smeared.

The lower plate LP may be fixed at a position, and as the upper plate UP is moved, the position of the lower plate LP relative to the upper plate UP may be varied. Alternatively, the lower plate LP may be moved in one direction such that the smear plate SP which is in contact with the lower plate LP may be slid for smearing the biological sample SA.

The lower plate LP may be formed of a transparent material. The smearing of the biological sample SA may be performed for optical observation of a thinly spread layer of the biological sample SA. For the optical observation of the biological sample SA and biological particles included in the biological sample SA, it may be preferable that the smear area of the biological sample SA and the lower plate LP be formed of a transparent material having a thickness less than a reference thickness.

The smear area and the external substrate ES included in the lower plate LP may be slide glass. Since the smear area is an area in which the biological sample SA is smeared, it may be preferable that the smear area cause low friction and be suitable for observing the biological sample SA.

### 3.3 Operation of Device

FIGS. 19 to 21 schematically illustrate a process of smearing a biological sample SA using the smear device according to an embodiment of the present disclosure. Hereinafter, the smearing of the biological sample SA using the smear device will be described with reference to FIGS. 19 to 21.

Referring to FIG. 19, according to an embodiment of the present disclosure, the smear device may bring an end of the smear plate SP supported by the upper plate UP into contact the smear area ES on the lower plate LP. In the smear device, the position at which the smear plate SP is brought into contact with the smear area ES may be spaced a predetermined distance from the sample SA in the smearing direction of the sample SA. Although the smear plate SP is shown as extending in a straight shape in FIGS. 19 to 21, at least a portion of the smear plate SP may be deformed according to the distance between the upper plate UP and the lower plate LP, the elasticity of the upper plate UP, and the inclination angle of the smear plate SP from the upper plate UP.

According to an embodiment of the present disclosure, the smear device may slide the smear plate SP to bring the smear plate SP into contact with the sample SA. The smear device may move the upper plate UP in a first direction D1 to slide the smear plate SP in the first direction D1. The smear device may move the upper plate UP in the direction D1 parallel to the direction in which the lower plate LP is arranged. The smear device may move the upper plate UP from a predetermined position by a predetermined distance in the first interval D1. In this case, the speed at which the upper plate UP is moved in a second direction D2 may be determined by considering the type of the sample SA and the shape of the smear plate SP.

The sample SA may be previously placed in the smear area ES. The sample SA may be applied to a position of the smear area ES by using a sample dripping unit separately provided to the smear device.

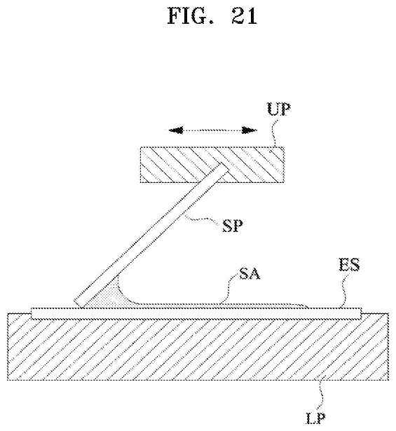

Referring to FIGS. 20 and 21, according to an embodiment of the present disclosure, the smear device may move the upper plate UP in the second direction

D2 such that the end of the smear plate SP supported by the upper plate UP may be slid in the second direction D2 opposite the first direction D1. As the upper plate UP is moved in the second direction D2, the smear plate SP may be slid in the second direction D2 such that the sample SA making contact with the smear plate SP may be smeared in the second direction D2. In this case, the speed at which the upper plate UP is moved in the second direction D2 may be determined by considering the type of the sample SA, the shape of the smear plate SP, or the like.

When the smear device slides the smear plate SP in the second direction D2, the sample SA making contact with the smear plate SP is moved in the second direction D2 while following the smear plate SP. As the sample SA is moved in the second direction D2 while following the smear plate SP, the sample SA may be smeared in the first direction D1. According to an embodiment of the present disclosure, the smear device may smear the sample SA on the external substrate by using the smear plate SP which has a inwardly recessed pattern surface. In this case, the phenomenon in which particles included in the sample SA are pushed outward may be mitigated owing to the pattern surface.

The smear device may stop moving the upper plate UP when the smear plate SP reaches a predetermined point on the smear area ES. The predetermined point may be determined by considering the length of the spreading area ES in the second direction D2.

According to another embodiment of the present disclosure, while the smear plate SP is slid in the second direction D2, the smear plate SP may be vibrated. As the smear plate SP vibrates, outward flows in the sample SA may be canceled. Therefore, the particles included in the sample SA may be more uniformly distributed. In another embodiment, while the smear plate SP is slid in the second direction D2, ultrasonic waves may be applied to the smear area ES to further mitigate the phenomenon in which the particles included in the sample SA are pushed outward.

In addition, although the volume and area of the sample SA are somewhat exaggerated in FIGS. 19 to 21, the sample SA may be smeared to form a single area in the smear area ES. In addition, the region in which the sample SA is distributed between the smear plate SP and the smear area ES may be limited to a region in which the smear plate SP and the smear area ES are very close to each other.

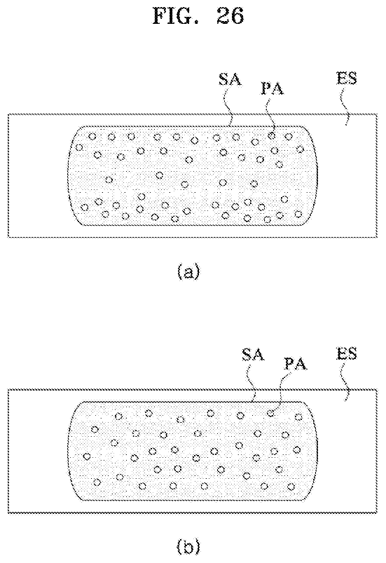

FIG. 26 is a view illustrating a sample SA which is smeared using a smear device and a smear plate according to an embodiment of the present disclosure. More specifically, FIG. 26 schematically illustrates the distribution of particles PA included in the sample SA according to the speed at which the smear plate having an inwardly recessed contact surface is slid on an external substrate ES by the smear device.

FIG. 26A illustrates a smeared state of the sample SA when the smear device slides the smear plate at a speed greater than a proper speed according to an embodiment of the present disclosure. When the smear plate is slid at a speed greater than a proper speed, the particles PA included in the sample SA may be relatively densely distributed in outer regions in the width direction of the external substrate ES. The heavier the particles PA, the more remarkably the outward dense distribution may occur. The outward dense distribution of the particles PA may occur because the influence of flow force exerted by the sample SA flowing along the inwardly recessed contact surface of the smear plate is less than the influence of lateral flow force and inertial force acting on the particles PA.

FIG. 26B illustrates a smeared state of the sample SA when the smear device slides the smear plate at a speed within an appropriate range. When the smear plate is slid at a speed within an appropriate range, the particles PA included in the sample SA may be uniformly distributed in the width direction of the external substrate ES. The particles Pa may be uniformly distributed because the influence of flow force exerted by the sample SA flowing along the inwardly recessed contact surface of the smear plate cancels the influence of lateral flow force and inertial force acting on the particles PA.

In this case, the proper sliding speed of the smear plate for uniformly distributing the particles PA contained in the sample SA may be determined by considering the type of the sample SA, the viscosity of the sample SA, the angle between the smear plate and the external substrate ES, the degree of concavity of the inwardly recessed contact surface of the smear plate, the shape of the inwardly recessed contact surface of the smear plate, or the like. In addition, the inclination angle of the smear plate from the external substrate ES, the degree of concavity of the inwardly recessed contact surface of the smear plate, and the shape of the inwardly recessed contact surface of the smear plate may be determined by considering the sliding speed of the smear plate on the external substrate ES.

### 3.4 Performing Smearing

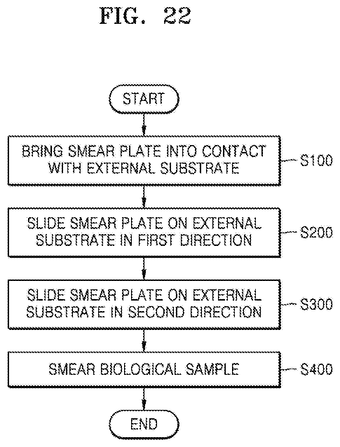

FIG. 22 is a flowchart illustrating a smear method according to an embodiment of the present disclosure. Referring to FIG. 22, the smear method according to the embodiment of the present disclosure is for smearing a biological sample in a smear area of an external substrate by using a smear plate, the smear method may include: bringing the smear plate into contact with the external substrate (S100); sliding the smear plate on the external substrate in a first direction (S200); sliding the smear plate on the external substrate in a second direction (S300); and smearing a biological sample (S400). According to the smearing method described below, smearing may be performed using the smear plate described above. In addition, the smear method may be performed by the smear device described above.

The bringing of the smear plate into contact with the external substrate (S100) may be performed by bringing the smear plate into contact with the external substrate at a predetermined inclination angle of the smear plate. In this case, a portion of the smear plate making contact with the external substrate may be inwardly recessed inward of the smear plate. The biological sample may be previously dripped on a smear area.

In the present embodiment, the smear plate may have a length and a width. The smear plate may have a pair of main surfaces. The smear plate may include a pattern surface to control the distribution of fine particles of the biological sample on the external substrate. In this case, the pattern surface may include a first point and a second point (closer to a midpoint of a body of the smear plate in the width direction of the body than the first point is to the midpoint of the body), and a first tangent line which is tangent to the pattern surface at the first point and is parallel to the main surfaces of the smear plate may have a smaller angle from the length direction of the smear plate than a second tangent line which is tangent to the pattern surface at the second point and is parallel to the main surfaces of the smear plate. A fine pattern including a plurality of concave and convex portions may be formed on the pattern surface.

In addition, the pattern surface may include: a first surface located on a left side in the width direction of the smear plate to guide fine particles toward a right side in the width direction of the smear plate; and a second surface located on the right side in the width direction of the smear plate to guide fine particles toward the left side in the width direction of the smear plate.

The sliding of the smear plate on the external substrate in the first direction (S200) may be performed to bring the smear plate into contact with the biological sample placed at a predetermined position in the smear area and thus to distribute the biological sample along the smear plate.

The sliding of the smear plate on the external substrate in the second direction (S300) may be performed to uniformly distribute the biological sample in the second direction opposite the first direction. The speed at which the smear plate is slid on the external substrate may be determined in consideration of the type of the biological sample, the viscosity of the biological sample, the temperature of the biological sample, the purpose of smearing, and the inclination angle of the smear plate.

The smearing of the biological sample (S400) may be performed by smearing the biological sample by using the smear plate to move the fine particles of the biological sample along the contact area toward the inside of the smear area in the width direction for mitigating the phenomenon in which the fine particles of the biological sample are relatively densely distributed in outer regions of the smear area in the width direction of the external substrate perpendicular to the second direction when the smear plate is slid in the second direction.

### 4. INSPECTION OF SAMPLE

The biological sample smeared using the smear plate or the smear device may be used for diagnosis or experiments. For example, a morphological diagnosis may be performed on a smeared blood sample. A person may be diagnosed by analyzing a blood sample of the person based on the morphology of white blood cells, the distributions of white blood cells, the number of red blood cells, or the like. For the analysis of the blood sample, the blood sample may be stained after the blood sample is smeared. In addition, for example, a smeared cell fluid sample may be inspected using an antigen-antibody reaction. To diagnose a target disease using a sample, the sample may be smeared and fixed, and a target protein related to the target disease may be detected from the sample using a fluorescent labeled antibody.

### 5. EXAMPLES

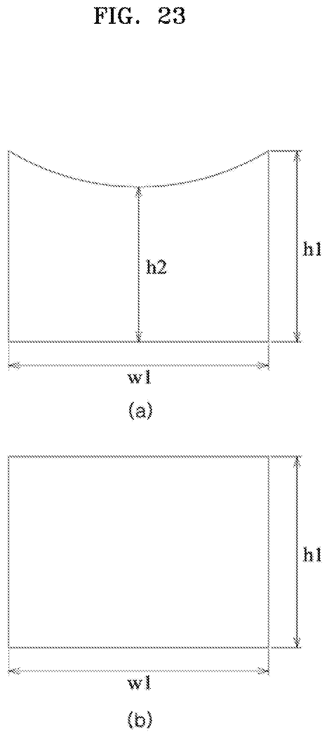

FIG. 23 is a schematic view illustrating smear plates used for smear experiments according to embodiments of the present disclosure. FIG. 23 shows a smear plate SP having an arc-shaped pattern surface and a smear plate SP′ having a flat end surface, which are used for smearing biological samples according to embodiments.

Referring to FIG. 23A, according to an embodiment of the present disclosure, a smear experiment may be performed using the smear plate SP having an arc-shaped pattern surface, a width w1 of 2.4 cm, a height h1 of 1.3 cm, and a center height h2 of 1.2 cm.

Referring to FIG. 23B, according to an embodiment of the present disclosure, a smear experiment may be performed using the smear plate SP′ having a width w1 of 2.4 cm, a height h1 of 1.3 cm, and a flat end surface, that is, a general pattern surface.

In the embodiments, each of the smear plates SP and SP′ forms an angle of 37° with a substrate on which blood is smeared. Furthermore, in the embodiments, each the smear plates SP and SP′ is slid on a blood smear substrate at a speed of 0.7 cm/s. However, when the smear method of the present disclosure is used, the angle between a smear plate and a blood smear substrate and the speed at which the smear plate is slid on the blood smear substrate may be varied.

Hereinafter, the results of blood smearing which is performed using the smear plate SP having an arc-shaped pattern surface and the smear plate SP′ having a general flat end surface will be described.

### 5.1 Example 1

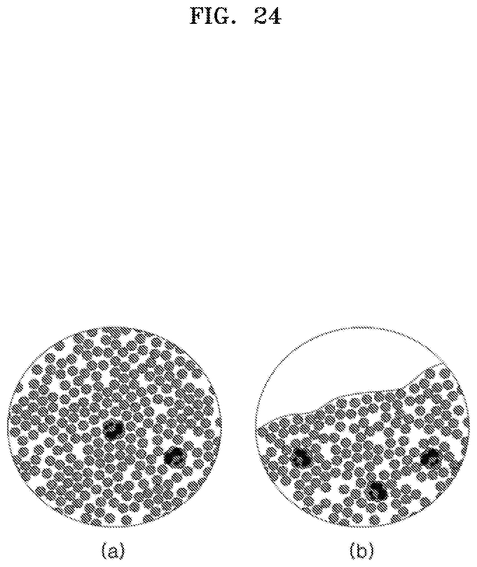

Here, an example in which smearing was performed using the smear plate SP shown in FIG. 23A is described. FIG. 24 is a view illustrating results of an experiment in which smearing was performed using the smear plate SP shown in FIG. 23A according to an embodiment of the present disclosure.

FIG. 24A is a schematic enlarged (×40) view illustrating a center region of a smear area when blood was smeared using the smear plate SP shown in FIG. 23A. FIG. 24B is a schematic enlarged (×40) view illustrating a side region of the smear area when blood was smeared using the smear plate SP shown in FIG. 23A.

Referring to FIG. FIG. 24, when blood is smeared using the smear plate SP having an arc-shaped pattern surface according to an embodiment of the present disclosure, the number of white blood cells observed in the center region (FIG. 24A) may be similar to the number of white blood cells observed in the side region (FIG. 24B).

### 5.2 Example 2

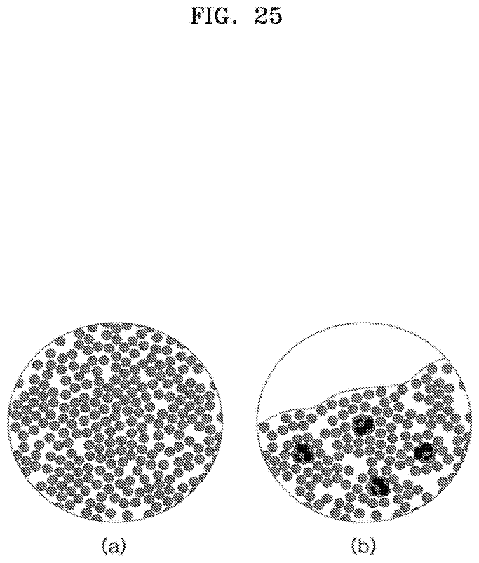

Here, an example in which smearing was performed using the smear plate SP′ shown in FIG. 23B is described. FIG. 25 is a view illustrating results of an experiment in which blood was smeared using the smear plate SP′ shown in FIG. 23B according to an embodiment of the present disclosure.

FIG. 25A is a schematic enlarged (×40) view illustrating a center region of a smear area when blood was smeared using the smear plate SP′ shown in FIG. 23B. FIG. 25B is a schematic enlarged (×40) view illustrating a side region of the smear area when blood was smeared using the smear plate SP′ shown in FIG. 23B.

Referring to FIG. 25, when blood is smeared using the smear plate SP′ having a flat end surface according to a method of the related art, the number of white blood cells may be greater in the side region (FIG. 25B) of the smear area than in the center region (FIG. 25A) of the smear area. The reason for this may be that when blood is smeared using the smear plate SP′ having a flat end surface, white blood cells are pushed outward in the smear area.

### 5.3 Conclusion

As described above with reference to FIGS. 24 and 25, when blood is smeared using the smear plate SP having an arc-shaped pattern surface according to an embodiment of the present disclosure, white blood cells may be uniformly distributed compared to the case in which blood is smeared by a method of the related art. In other words, when smearing is performed using a smear plate manufactured according to an embodiment of the present disclosure, particles included in a sample may be uniformly distributed compared with the case of smearing blood by a method of the related art.

The above descriptions of technical ideas of the present disclosure are merely examples, and it will be apparent to those skilled in the art that modifications and variations could be made without departing from the scope of the present disclosure. Therefore, the embodiments of the present disclosure described above may be implemented individually or in combination.

The embodiments of the present disclosure are for illustrative purposes only and are not intended to limit the scope of the present disclosure. The scope of the present disclosure should be construed according to the appended claims, and it should be understood that all technical ideas equivalent to those described above are within the scope of the present disclosure.

1. A smear plate for smearing a biological sample on an external substrate, the smear plate comprising:
a body having a plate shape with a length, a width, and a pair of main surfaces, the body having a predetermined angle with respect to the external substrate;
a support portion located on a side of the body and spaced apart from the external substrate by a predetermined distance; and
a contact portion located on an opposite side of the body and comprising a pattern surface which is recessed inward of the body to induce fine particles included in the biological sample to move toward a midpoint in a width direction of the body for preventing the fine particles from being relatively densely distributed in outer regions in a width direction of the external substrate.

2. The smear plate of claim 1, wherein
an angle between a length direction of the body and a first tangent line, which is tangent to the pattern surface at a first point and is parallel to the main surfaces of the body, is less than an angle between the length direction of the body and a second tangent line, which is tangent to the pattern surface at a second point and is parallel to the main surfaces of the body, and
the second point is closer to the midpoint in the width direction of the body than the first point is to the midpoint.

3. The smear plate of claim 1, wherein the pattern surface comprises:
a first surface located on a left side in the width direction of the body to guide the fine particles toward a right side in the width direction of the body; and
a second surface located on the right side in the width direction of the body to guide the fine particles toward the left side in the width direction of the body.

4. The smear plate of claim 1, wherein
the pattern surface has a circular arc shape.

5. The smear plate of claim 1, wherein
the pattern surface has a triangular shape with a vertex at the midpoint in the width direction of the body.

6. The smear plate of claim 1, wherein
the pattern surface comprises a plurality of concave and convex portions.

7. The smear plate of claim 1, wherein,
when the smear plate is brought into contact with the external substrate and smears the biological sample, an inclination angle of at least a region of the contact portion from the external substrate is different from an inclination angle of the support portion from the external substrate.

8. The smear plate of claim 1, wherein,
when the smear plate is brought into contact with the external substrate and smears the biological sample, an inclination angle of the contact portion from the external substrate varies according to positions of the contact portion in the width direction of the body.

9. The smear plate of claim 1, wherein
an inclination angle of the contact portion from the external substrate decreases in outward directions parallel to the width direction of the body.

10. The smear plate of claim 1, wherein
the biological sample is blood, and the fine particles are blood cells included in the blood.

11. The smear plate of claim 1, wherein
the contact portion is at least partially hydrophilic and comprises a lower surface which is brought into contact with the biological sample.

12. The smear plate of claim 1, wherein
the body has elasticity.

13. A smear device for smearing a biological sample, the smear device comprising:
an upper plate configured to support a smear plate at a predetermined downward inclination angle with respect to a first direction in which the biological sample is smeared, move in a second direction opposite the first direction to slide the smear plate for bringing the smear plate into contact with the biological sample, and move in the first direction to slide the smear plate such that the biological sample making contact with the smear plate is smeared in the first direction while following the smear plate; and
a lower plate spaced downward from the upper plate by a predetermined distance and comprising a smear area in which the biological sample is placed and smeared,
wherein an end surface of the smear plate to be brought into contact with the smear area is recessed in the first direction such that when the smear plate is brought into contact with the smear area and smears the biological sample in the first direction, outward movement of fine particles included in the biological sample is reduced.

14. The smear device of claim 13, wherein
an angle between a length direction of a body of the smear plate and a first tangent line, which is tangent at a first point to the end surface of the smear plate to be brought into contact with the smear area and is parallel to main surfaces of the body, is less than an angle between the length direction of the body and a second tangent line, which is tangent at a second point to the end surface of the smear plate and is parallel to the main surfaces of the body, and
the second point is closer to a midpoint in a width direction of the body than the first point is to the midpoint.

15. The smear device of claim 13, wherein
each of the smear plate and the smear area has a length and a width, and
the end surface of the smear plate to be brought into contact with the smear area comprises:
a first surface located on a left side in a width direction of the smear plate to guide the fine particles toward a right side in a width direction of the smear area; and
a second surface located on the right side in the width direction of the smear plate to guide the fine particles toward the left side in the width direction of the smear area.

16. The smear device of claim 13, wherein
the end surface of the smear plate to be brought into contact with the smear area comprises a plurality of concave and convex portions.

17. The smear device of claim 13, wherein
the smear plate comprises a contact portion having an inclination angle varying with respect to the lower plate when being brought into contact with the lower plate, and
when the smear plate is brought into contact with the smear area and smears the biological sample, the inclination angle of at least a region of the contact portion with respect to the lower plate is different from an inclination angle of a support portion of the smear plate with respect to the lower plate.

18. The smear device of claim 13, wherein,
when the smear plate is brought into contact with the smear area and smears the biological sample, an inclination angle of a contact portion of the smear plate from the lower plate varies according to positions of the contact portion in a width direction of a body of the smear plate.

19. The smear device of claim 13, wherein
the biological sample is blood, and the fine particles are blood cells included in the blood.

20. A smear method of smearing a biological sample in a smear area of an external substrate by using a smear plate, the method comprising:
bringing the smear plate into contact with the external substrate at a predetermined inclination angle with respect to the external substrate;
sliding the smear plate on the external substrate in a first direction such that the smear plate is brought into contact with the biological sample placed at a predetermined position in the smear area and the biological sample is distributed along the smear plate;
sliding the smear plate on the external substrate in a second direction opposite the first direction such that the biological sample is uniformly distributed in the second direction; and
smearing the biological sample by using the smear plate while mitigating a phenomenon, in which fine particles included in the biological sample are relatively densely distributed in outer regions of the smear area in a width direction of the external substrate perpendicular to the second direction when the smear plate is slid in the second direction, by inducing the fine particles to move inward in the smear area along a contact area in the width direction of the external substrate,
wherein an area of the smear plate making contact with the external substrate is concavely recessed inward of the smear plate.

21. The smear method of claim 20, wherein
the smear plate has a length, a width, and a pair of main surfaces,
the smear plate comprises a pattern surface to control distribution of the fine particles on the external substrate, and
the pattern surface comprises a first point and a second point, wherein a first tangent line which is tangent to the pattern surface at the first point and is parallel to the main surfaces of the smear plate has a smaller angle with respect to a length direction of the smear plate than a second tangent line which is tangent to the pattern surface at the second point and is parallel to the main surfaces of the smear plate, and the second point is closer to a midpoint in a width direction of a body of the smear plate than the first point.

22. The smear method of claim 21, wherein the pattern surface comprises:
a first surface located on a left side in a width direction of the smear plate to guide the fine particles toward a right side in the width direction of the smear plate; and
a second surface located on the right side in the width direction of the smear plate to guide the fine particles toward the left side in the width direction of the smear plate.

23. The smear method of claim 21, wherein
the pattern surface comprises a plurality of concave and convex portions.

24. The smear method of claim 20, wherein
the biological sample is blood, and
the fine particles are blood cells included in the blood.
